(19) United States
(12) Patent Application Publication
(10) Pub. No.: US 2012/0100608 A1
(43) Pub. Date: Apr. 26, 2012

(54) LYMPHOCREME

(76) Inventors: KEVIN PATRICK SLATTERY, Smithtown, NY (US); Colleen Margaret Slattery, Garden City, NY (US)

(21) Appl. No.: 12/894,908

(22) Filed: Oct. 25, 2010

(51) Int. Cl. C12N 5/02

(52) U.S. Cl. 435/375

(57) ABSTRACT

The mixture is a topical crème for use in the treatment of cutaneous T-cell Lymphoma.

The mixture is composed of

Retin A Crème 0.05% 20 G(Grams); Hydrocortisone Crème 1% 20 G(grams); Vitamin D 30 gtts(drops) 400 iu.

1. The mixture when applied directly to T-cells inhibits their growth.

2. The mixture when applied directly to T-cells induces regression.
